(19) United States
(12) Patent Application Publication
(10) Pub. No.: US 2003/0158237 A1
(43) Pub. Date: Aug. 21, 2003

(54) Combination of antioxidant substances for the treatment of alzheimer's disease

(75) Inventors: Uri Saragovi, Montreal (CA); Joseph Bassili, Montreal (CA); Khassam Elfarrah, Dollard-des-Ormeaux (CA)

Correspondence Address: MERCHANT & GOULD PC, P.O. BOX 2903, MINNEAPOLIS, MN, 55402-0903, US

(73) Assignee: Colba R & D Inc., St-Laurent (CA)

(21) Appl. No.: 10/235,613

(22) Filed: Sep. 4, 2002

(30) Foreign Application Priority Data
Sep. 4, 2001 (CA) 2,357,053

(51) Int. Cl. A61K 31/455; A61K 31/353; A61K 31/192; A61K 31/355; A61K 31/235

(52) U.S. Cl. 514/356; 514/456; 514/458; 514/570; 514/533

(57) ABSTRACT

Disclosed is a pharmaceutical composition for the treatment of cognitive symptoms caused by Alzheimer's disease in a mammal. This composition comprising vitamin E, quercitin, caffeic acid, nicotinic acid or derivatives and/or analog thereof, and a pharmaceutically acceptable excipient.

### FIELD OF THE INVENTION

The present invention relates to the use of a combination of antioxidant substances for the treatment of cognitive disorders such as Alzheimer's disease (AD).

### BACKGROUND OF THE INVENTION

Alzheimer's disease (AD) is the most common of dementias. Its frequency, in later years, has dramatically increased due to the rise of life expectancy. This disease represents, according to different statistical data, from 50 to 75% of all dementias. Etiological hypotheses of AD such as those based on amyloid hypothesis, cholinergic hypothesis, oxidative hypothesis, genetic hypothesis and immunologic hypothesis are numerous and complex. It is considered that the trigger of the disease is a combination of them, and particularly of genetic and neurobiologic processes that take place simultaneously.

Amyloid and AD

The patient's brain with Alzheimer's disease is invaded by a protein known as beta-amyloid peptide (beta A4). Beta A4 is part of the senile plaques that together with the neurofibrillary degeneration constitute the histopathology of AD, of other dementias and of the aging process depending on the density of the injury.

The Amyloid Precursor Protein (APP), codified in chromosome 21, has 695 to 770 amino-acid deposits, being a normal constituent of the neuronal membranes. The formation of protein beta A4 requires the cleavage of the APP, which is expressed by numerous types of cells. The beta-secretase cleaves an end of the APP, while a second secretase, known as gama secretase, cleaves the other end of A beta. It is a very difficult task to identify these divisory enzymes (beta and gama secretase) since the cells contain hundreds of these protease types. Therefore, the applied strategy to find these enzymes was based on identifying their genes.

The beta-secretase in isolation has been called Beta-site APP-Cleaving Enzyme (BACE). It is an enzyme that can cleave the APP, and it diminishes the production of A beta in cell cultures. Its levels are higher in the neurons than in the glia, supporting the hypothesis that the neurons are the first extracellular source of beta A4 deposited in amyloid plaques.

According to Dennis Selkoe et al, the preseniline 1, involved in so me hereditary forms of AD, could be identified with the gama-secretase. Nowadays, it is considered that this enzyme plays a secondary role.

The beta A4 would cause cell death via apoptosis, for disruption in the homeostasis of calcium, activating calcium channels and second messengers like the protein kinases which provoke nerve cell death.

Oxidative Stress and AD

The oxygen free radicals have been involved in the etiology and the consequences of different diseases, including AD. The aging processes, brain injury and ischemia are related to the production of free radicals.

There are several hypotheses regarding the mechanisms that trigger oxidative stress in AD, that is to say, the damage produced by an increase of the free radicals activity on the biomolecules and, in the case of the central nervous system, predominantly on the lipids. The excess of free oxygen alters the ionic balance of calcium between the neurons and its mitochondrias. The beta amyloid protein seems to be involved in the oxidative stress mechanism. The increased presence of this peptide is related to an important production of free radicals and subsequent cell damage. At the same time, the lipid peroxidation of the membrane may increase the vulnerability of the APP transmembrane to the abnormal cleavage by the proteases related to the deposit of BA4, and for this reason the BA4 increases.

Postmortem exams of cerebral cortex of patients with AD were compared to brain tissue of patients that died without having antecedents of the neurodegenerative disease. In the first ones there was a significant increase of lipid peroxidation measuring dialdehyde (TBARS and malondialdehyde).

Some authors have determined the oxidative stress by chemiluminescence (a technique by means of which some components of lipid peroxidation are detected as they have the peculiarity of liberating energy by means of photons and whose quantification determines the magnitude of the damage made to the lipids).

In a study made by Barkats M, et al in the Hospital de la Pitie Salpetriere, in France, published in Neurochemist,(4), the authors utilized a strategy of gene transfer to increase the antioxidant potential of nerve cells before exposing them to toxic fragments of A-beta. More precisely, they evaluated if the intracellular overexpression of glutathione peroxydase (GPx) could increase the resistance of cells PC12 of pheochromocytoma and of the embryonic neurons of the cortical area of rats against the toxicity of the A-beta. As the adenovirus is an efficient vector for the transfer of genes on postmiotic cells both in vitro as on alive cells, it has been used to increase intracellular expression of GPx which is the main captor at brain cell level of the peroxide of hydrogen radical. The cells infected this way were then exposed to toxic concentrations of A-beta. Both PC12 cells and the cortical ones infected with Ad-GPx have been significantly more resistant to A-beta exposure. This information strengthens the hypothesis of the role of the hydrogen peroxide in the toxicity mechanism by A-beta injury.

The increase of glutamate, which is present in dementias, produces an excessive opening of the calcium channels and this produces a slow, toxic increase of the intraneural calcium. This factor activates enzymatic and metabolic processes with lipid peroxidation and formation of free oxygen radicals. The free radicals wreck the organelles and the neuron membranes, contributing in this way to the neurodegenerative process.

Other reports have pointed out possible alterations in the availability or quality of the antioxidant enzymes. The results have not been conclusive, or rather they suggest that the neuronal death mechanism, if it is produced by oxidative stress, is not due to deficiency of these enzymes, at least in the cerebral cortex.

Schippling et al measured the lipoprotein oxidation in the cerebrospinal fluid and in the plasma of 29 patients with AD. They found that such oxidation was significantly high in comparison with strict controls. In the same way, the ascorbic levels were low in patients with AD, but not those of alpha-tocopherol.

The presence of oxidative stress has not only been detected by the increase of lipid peroxidation through the different mentioned techniques, but also by the decrease in the activity of antioxidant enzymes, particularly the superoxide dismutase (SOD).

Immunological Activity and AD

In Alzheimer's disease there is an immunological hyperactivity. The cytokines (interleukins 1 and 6), produced by the increase of the microglial cell activity when amyloid is deposited in the neurons, increase in plasma. Alpha-antichymotrypsin and alfa-2 macroglobulin also increase.

It is believed that cytokines would influence in the regulation of the gene expression of the APP and/or in the proteolytic process, contributing to the production of oxygen free radicals. That is the reason of the importance of some studies carried out with non-steroidal anti-inflammatory drugs.

The inflammatory response makes that the astrocytes become part of the plaques. Recently, it has been established that there is a relationship between the apolipoprotein E4 and the TBARS levels (products of lipid lipoperoxidation). This protein that degrades the amyloid substance is made based on an anomaly codified in chromosome 19. This anomaly makes the amyloid not to degrade appropriately and to deposit it in the neurons.

Apolipoprotein E plays an important role in the transport, generation and cleareance of lipids. It has to do with the myelinization and with the neuroplasticity. The anomaly of apolipoprotein E-4 (APOE-4) is codified in chromosome 19, related with the AD of late beginning.

APOE-4 is a physiological transporter of cholesterol that also intervenes in the myelinization, in the neuroplasticity and in the deposit of amyloid. It is present in the senile plaques, in the neurofibrillary degeneration and in the cerebrovascular amyloid. There are studies that evidence the neurotrophic, immunomodulatory and antioxidant functions of the APOE. The APOE-4 is nowadays considered as one of the recognized risk factors for the development of the Alzheimer's disease.

Genetic Hypothesis of AD

Other biomolecules like proteins and the DNA nucleotides suffer damage in their molecular structure in the brain of patients with AP. The carbonyl protein, one of the components of protein oxidation, is significantly increased in the hippocampus and in the inferior parietal lobe of these patients.

One of the methods most commonly used to evaluate the oxidative alteration of the molecular structure is the measurement of an oxidative component of one of its bases: the 8-hydroxy-2-deoxyguanosine. This substance increases in the brain in the aging process, particularly the one coming from mitochondrial DNA. In patients suffering from AD, this increase triples, indicating an oxidative stress that also affects the DNA. The oxidative stress with hemi-oxygenase 1 is superior in neurons and astrocytes of cortex and hippocampus.

Another theory is the presence of an increase in the iron deposits in the cerebral cortex of these patients. As it is known, iron is an important catalyst of free radicals and it activates the lipid peroxidative processes.

Another evidence of the presence of an important amount of oxidative stress in these patients is the so called Products of Advanced Glycosylation (PAG), formed by non enzymatic reactions of glucose with earlier deposits of proteins which are potentially toxic for the cells. The PAG are usually produced by an accelerated oxidation of the glycoproteins and it has been observed that they are increased in the senile plaques of the patients with AD.

### SUMMARY OF THE INVENTION

A first object of the present invention concerns a combination of specific and known antioxydants used for the treatment of cognitive symptoms caused by the Alzheimer's disease.

More precisely, the combination comprises vitamin E, quercitin, caffeic acid nicotinic acid and derivatives and/or analogs thereof.

A second object of the present invention concerns a pharmaceutical composition comprising the above-mentioned combination of antioxidants.

A third object of the invention concerns a method for treating Alzheimer's disease by the administration of the pharmaceutical composition comprising the combination of antioxidants of the present invention.

### BRIEF DESCRIPTION OF THE DRAWINGS

FIG. 1 is a schematic representation of the oxidative stress pattern in AD.

FIG. 2 shows the behavior of the average scores during the entire time period for the different groups for the ADAS Test.

FIG. 3 shows the behavior of the average scores during the entire time period for the different groups for the MMSE Test.

FIG. 4 shows the behavior of the average scores during the entire time period for the different groups for the Hamilton Test.

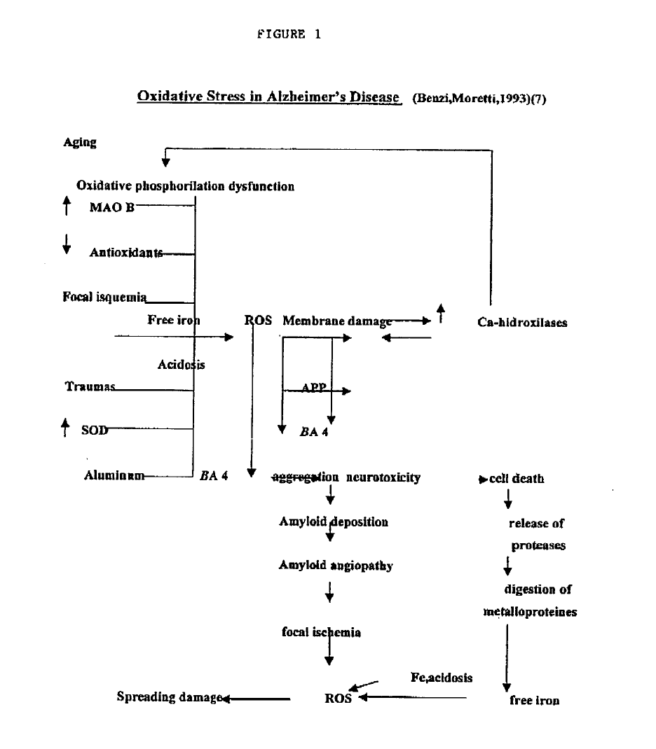

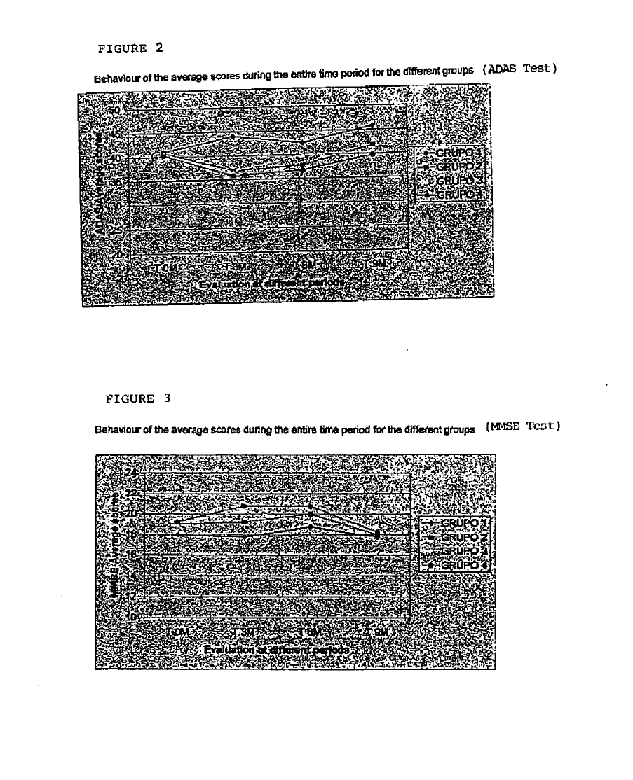

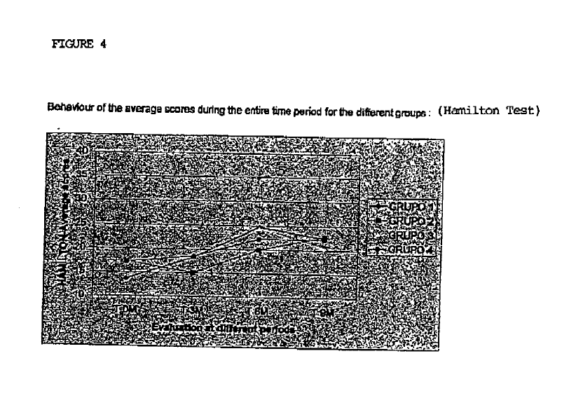

### DETAILED DESCRIPTION OF THE INVENTION

The present invention relates to a pharmaceutical composition which comprises a combination of antioxidants selected from the group consisting of vitamin E, quercitin, nicotinic acid and caffeic acid.

Other antioxidants such as selegiline, idebenone, oestrogens, antlinflammatory products, ginkgo bilova, ascorbic acid, beta carotene, melatonin, coenzyme Q and phenolic compounds can also be used in the pharmaceutical composition.

According to the present invention, it is possible to use from 0.1 to 100 mg of vitamin E, quercitin, nicotinic acid and caffeic acid and from 500 to 2500 UI of vitamin E. In a preferred embodiment, the following dose is used: 50 mg of quercitin; 50 mg of caffeic acid; 50 mg nicotinic acid and 1000 UI264 of vitamin E.

Preferably, RRR-α-tocopherol (d-alpha tocopherol) is used as the vitamin E; quercitin dihydrate (3,3′,4′5,7 pentahydroxyflavone.is used as the quercitin; and (3,4-dihydroxycinnamic) acid is used as the caffeic acid. No preferred form of nicotinic acid is used.

The pharmaceutical composition of the present invention is best administered to a patient in the solid form such as in a capsule. In the solid form, vitamin E is more stable (to temperature and light). In liquid form, vitamin E is less stable and its life time is shorter. Furthermore, in this later form, the efficacy of vitamin E cannot be guaranteed for a long period. Furthermore, in the liquid form, vitamin E may be insoluble when mixed with other antioxydants or chemical compounds.

Vitamin E

Vitamin E is an essential nutrient that functions as an antioxidant in the human body. It is essential, by definition, because the body cannot manufacture its own vitamin E and thus it must be provided by foods and supplements.

Vitamin E is a generic term that includes all entities that exhibit the biological activity of α-Tocopherol. In nature, eight substances have been found to have vitamin E activity. They are defined by four tocopherols and four tocotrienols. The tocopherols are alpha-tocopherol, beta-t ocopherol, gamma-tocopherol and delta-tocopherol. The tocotrienols are the alpha-tocotrienol, beta-tocotrienol, gamma-tocotrienol and delta-tocotrienol.

The vitamin E derivatives or related compound and analogs are potent antioxidants that block the lipid peroxidation by donation of hydrogen to the peroxidated lipids. The central nervous system is especially vulnerable to lipid peroxidation.

It has further been observed that vitamin E blocks the neurotoxic effects of the free radicals produced by excitotoxicity and it improves the performance altered by old age.

Thus according to the present invention, any chemical entities exhibiting the biological activity of α-tocopherol is meant to encompassed by the definition of vitamin E.

Alternate names for vitamin E include Alpha Tocopherol, D-Alpha-Tocopherol, D-Beta-Tocopherol, D-Delta-Tocopherol, D-Gamma-Tocopherol, D-Tocopherol, DL-Alpha-Tocopherol, DL-Tocopherol, Mixed Tocopherols, Tocopheryl Acetate, Tocopheryl Succinate.

Other vitamin E related compounds may also be used in the context of the present invention. They may be selected from the group consisting of vitamin E acetate, DL-α-Tocopherol succinate, d-alpha-Tocopherol acetate, (+)-alpha-Tocopherol, mixed isomers thereof, (+)-alpha-Tocopherol and mixed isomers, (+)-alpha-Tocopherol acid succinate, (+)-alpha-Tocopherol acetate, (+)-alpha-Tocopherol, (±)-alpha-Tocopherol nicotinate, (±)-alpha-Tocopherol acetate, (±)-alpha-Tocopherol, DL-α-Tocopherol acetate, (±)-alpha-Tocopherol phosphat e Disodium Salt, (+)-α-Tocopherol, (±)-6-Hydroxy-2,5,7,8-tetramethylchromane-2-carboxylic acid, (R)-Trolox™ methyl ether, (S)-Trolox™ methyl ether, 2-(alpha-D-glucopyranosyl)methyl-2,5,7,8-tetramethylchroman-6-ol, 2,2,5,7,8-pentamethyl-6-chromanol (PMC), 2,3-dihydroxy-3,3-enono-1,4-lactone and chromone residues, α-Tocophenol hydroquinone, α-Tocophenol quinone, Vitamin CE(5a-tocopheryl ascorbate) and 2-(α-D-glucopyranosyl)methyl-2,5,7,8-tetramethyl chroman-6-Ol.

The vitamin E analogs may be selected from the group consisting of: Ascorbic acid, Beta-Caroten, Butylated-Hydroxy-Toluene, Butyated-Hydroxy-Anisol, Calcium Citrate, Canthaxanthin, Melatorin, Nordihydroguaiaretic acid, Propyl Gallate, Selenium, Silymarin, Sulfur Dioxide, Thioctic Acid.

The acetate and succinate derivatives of the natural Tocopherols have vitamin E activity, as do synthetic tocopherols and their acetate and succinate derivatives. Of these, d-alpha-tocopherol (RRR-alpha-tocopherol) has the highest bioavailability and is the standard against which all the others must be compared.

Natural and synthetic vitamins E are not equivalent in composition, structure and/or bioavailability. Natural vitamin E (RRR-alpha-tocopherol or di-alpha-tocopherol) is a single entity. Synthetic vitamin E (al l-rac-alpha-tocopherol or di-alpha tocopherol) is a mixture of eight stereoisomers in equal amounts.

As mentioned above, vitamin E is recognized to be the major antioxidant in lipid body tissues and the primary defence against lipid peroxidation-neutralizing free radicals, terminating chain reactions and limiting free radical/oxidative damage. Vitamin E is particularly important in tissues that contain relatively high levels of polyunsaturated fatty acid (brain and central nervous system) and in those that are in contact with oxygen (lung), providing protection for microsomes and mitochondria.

In rats and in cell cultures it slows down damage and neuronal death even in cells with amyloid deposit.

In cell cultures and in animals, it reduces neurotoxicity and neuronal death. The hydrogen peroxide and the free radicals produced by beta A4 to cultured neurons and endotelial cells is blocked by vitamin E and other antioxidants. Thus, vitamin E reduces beta A4 induced cell death in hippocampal cell cultures. Vitamin E also improves cognitive performance in aged animals and reduces degeneration of hippocampal cells following cerebral ischemia.

Vitamin E status in EA is controversial. A study found increased vitamin E levels suggesting a possible compensatory response to oxidative stress, another found no difference with controls, and another found decrease of vitamin E in AD. The range of daily dose goes between 200 and 3,000 UI, with an average range of 400 to 1,000 UI.

Adverse symptoms are uncommon: cataract, haemorrhage risk in patient with vitamin K deficiency, and syncope. It would have protective effects on the immunologic response and cardiac diseases. With high doses (3000 IU), appear indigestion, gastric distress, diarrhea and severe cramps.

In a multicenter comparative double blind study of 2 years of duration with selegiline (10 mg daily), vitamin E and their combination, with 341 patients, significantly positive results were obtained with the vitamin E (2,000 UI/daily) and the selegiline in single-agent therapy; but not with their combination. A retarded cognitive deterioration of 25% was observed for both drugs, as well as the delay in institutionalisation of the patients due to an improvement of the global function.

Caffeic Acid

Caffeic acid also known as 3-(3,4-Dihydroxyphenyl)-2-propenoic acid, found in many fruits, vegetables, seasonings and beverages consumed by humans, principally in conjugated forms such as chlorogenic acid.

Its action mechanism is not completely known, but the caffeic acid would have a beneficial effect in atherosclerosis, inflammation, neurodegenerative dysfunctions and acquired immunodeficiency syndromes.

Equivalents of such a compound may be selected from the group consisting of 5(4)-(2-Carboxyethenyl)-1,2-dihydroxybenzene; 4-(2′-Carboxyvinyl)-12-dihydroxybenzene; 3,4-Dihydroxybenzeneacrylic acid; 3,4 Dihydroxycinnamic acid; 3-(3,4-Dihydroxyphenyl)propenoic acid; 3,4-Dihydroxyhydrocinnamic acid; o-Coumaric acid; p-Coumaric acid; and Caffeic acid phenethyl ester.

The effects of phenolic compounds like the caffeic acid, present in the olive oil, over the oxidation of the low density protein (LDL) have been investigated. This process plays an important role in atherosclerosis, through the alteration of intra-cell signals in the vessel wall. The antioxidant effect of the caffeic acid is protective. When 0.025-0.3 mg/l of caffeic acid is added to isolated LDL, its time of oxidation is prolonged in a dose dependent mechanism.

Recently the action of the caffeic acid has been studied compared with that of the N-acetylcystein, that of the acetate d-alpha-tocopherol, and that of the ascorbic acid in the modulation of signal transduction related with the apoptosis induced by the ceramide. The ceramide acts as a second messenger in signal transduction produced by the stress or extracellular agents. It was observed that the caffeic acid inhibits significantly, in comparison with the other compounds, the activity of the NF-kappa B binding induced by ceramide and the apoptotic response by its antioxidant effects.

Another hypothesis on its action mechanism, apart from its antioxidant effect, is the inhibition of the protein tyrosine kinase activity, and this would inhibit the apoptosis.

Nicotinic Acid

Niacin and niacinamide function in the biochemistry of humans and other organisms as components of the two coenzymes: nicotinamide adenine dinucleotide (NAD) and nicotinamide adenine dinucleotide phosphate (NADP). These operate in many enzyme-catalysed oxidation and reduction reactions. The deficiency state in humans causes skin disease, diarrhea, dementia, and ultimately death. Lean meats, peanuts and other legumes, and whole-grain or enriched bread and cereal products are among the best sources of niacin.

Nicotinic acid and nicotinamide, commonly called niacin, are the dietary precursors for NAD(+) (nicotinamide adenine dinucleotide), which is required for DNA synthesis, as well as for the activity of the enzyme poly (ADP-ribose) polymerase-1 for which NAD(+) is the sole substrate. This enzyme is highly activated by DNA strand breaks during the cellular genotoxic stress response and is involved in base excision repair.

In vitro as well as animal studies indicate that niacin deficiency increases genomic instability especially in combination with genotoxic and oxidative stress.

Studies suggest that nicotinamide can be considered as a potent antioxidant capable of protecting the cellular membranes in brain, which is highly susceptible to prooxidants, against oxidative damage induced by reactive oxygen species (ROS).

In animal studies, it had been observed that nicotinamide showed significant inhibition of oxidative damage induced by ROS in rat brain mitochondria. In a study with the tertiary butylhydroperoxide-(t-BuOOH) treated mouse was used as a model to study the oxidative stress that is associated with various neurodegenerative diseases. The results directly implicate DNA damage in apoptosis and necrosis. Nicotinamide was able to prevent DNA fragmentation induced by low-dose t-BuOOH.

Other authors observed that nicotinamide is a robust neuroprotective agent against ischemia/reperfusion-induced brain injury in rats, even when administered up to 2 hours after the onset of stroke: nicotinamide improved both anatomic and functional indices of brain damage.

Nicotinic acid also known as pyridine 3-carboxylic acid and analogs thereof may be used in the combination of the present invention for the treatment of AD.

To this day, more than 233 different compounds of nicotinic acid are known. Each of them may be used in the context of the present invention. More particularly, the nicotinic acid compounds may be selected from the group consisting of: Nicotinic acid, Isonicotinic acid, 3-Pyridinecarbonitrile, 2,4,5,6-Tetrachloro-3-pyridinecarboxylic acid, 2,6,-Dichoro-5-fluoro-3-pyridinecarboxylic acid, 2-Chloro-6-methylnicotinic acid, 5-Bromonicontinic acid, Arecoline Hydrobromide, NO-711 Hydrochloride and Nicotinic acid ethyl ester, nicotinic acid (1-4-bromo-phenyl)-propylidene)-hydrazide, nicotinic acid (1-(4-pentyl-phenyl-ethylidene)-hydrazide, nicotinic acid (1-benzyl-propylidene)-hydrazide, nicotinic acid (1-methyl-5-nitro-2-oxo-1,2-dihydro-indol-3-ylidene)-hydrazide, nicotinic acid (1-thiophen-2-yl-ethylidene)-hydrazide, nicotinic acid (2,4-dichloro-benzylidene)-hydrazide, nicotinic acid (2-chloro-benzylidene)-hydrazide, nicotinic acid (2,4-dihydroxy-benzylidene)-hydrazide, nicotinic acid (2,4-dimethoxy-benzylidene)-hydrazide, nicotinic acid (2-bromo-3-phenyl-allylidene)-hydrazide, nicotinic acid (2-hydroxy-benzylidene)-hydrazide, nicotinic acid (2-methyl-3-phenyl-allylidene)-hydrazide, nicotinic acid (2-trifluoromethyl-benzylidene)-hydrazide, nicotinic acid (3,4,5-trimiethoxybenzylidene)-hydrazide, nicotinic acid (3,5-di-tert-butyl-4-hydroxy-benzylidene)-hydrazide, nicotinic acid (3,5-dibromo-2-hydroxy-benzylidene)-hydrazide, nicotinic acid (3-ethoxy-hydroxy-benzylidene)-hydrazide, nicotinic acid (3-nitro-benzylidene)-hydrazide, nicotinic acid (3-phenyl-allylidene-hydrazide, nicotinic acid (4-bromo-benzylidene)-hydrazide, nicotinic acid (4-chloro-benzylidene)-hydrazide, nicotinic acid (4-dimethylamino-benzylidene)-hydrazide, nicotinic acid (4-hydroxy-benzylidene)-hydrazide, nicotinic acid (4-isopropyl-benzylidene)-hydrazide, nicotinic acid (4-methyl-benzylidene)-hydrazide, nicotinic acid (4-nitro-benzylidene)-hydrazide, nicotinic acid (5-indanylmethylene)-hydrazide, nicotinic acid n′-(1,1-dioxo-tetrahydro-thiophen-3-yl)-n′-phenyl-hydrazide, nicotinic acid n′-(4-methoxy-benzoyl)-hydrazide, nicotinic acid n′-phenoxyacetyl-hydrazide, nicotinic acid naphthalen-1-ylmethylene-hydrazide, nicotinic acid 0-tolyl ester, nicotinic acid p-tolyl ester, nicotinic acid pyridin-3-ylmethylene-hydrazide, nicotinic acid thiophen-2-ylmethylene-hydrazide, 1-bicyclo (2.2.1) hept-2-yl-ethylamine, nicotinic acid benzo (1,3)dioxol-5-ylmethylene-hydrazide, nicotinic acid benzylidene-hydrazide, nicotinic acid fluoren-9-ylidene hydrazide, nicotinic acid (4-diethylamino-benzylidene)-hydrazide, nicotinic acid, 1,2-diphenylethylammonium salt, nicotinic acid, 2-amino 1-(4-nitro-phenyl)-ethanol, nicotinic acid, octadecylamine salt, nicotinic acid (2,4-dimethoxy-benzylidene)-hydrazide, nicotinic acid (2-bromo 3-phenyl-allylidene)-hydrazide, nicotinic acid (2-chloro-benzylidene) hydrazide, nicotinic acid hydroxamate, nicotinic acid mononucleotide, nicotinic acid n-oxide, 1-methylnicotinamide chloride salt, 1-methylnicotinamide iodide salt, and nicotinic acid adenine dinucleotide sodium salt.

Isonicotinic acid salts may also be used in the combination of the present invention for the treatment of AD. To this day, 86 different compounds of isonicotinic acid salts are known. More particularly, these may be selected from the group consisting of: Isonicotinic acid ((4-nitro-phenyl)-phenyl-methylene)-hydrazide; Isonicotinic acid (1-allyl-2-oxo-1,2-dihydro-indol-3-ylidene)hydrazide; Isonicotinic acid (1-benyl-2-oxo-1,2-dihydro-indol-3-ylidene)hydrazide; Isonicotinic acid (1-benzyl-propylidene)-hydrazide; Isonicotinic acid (1-methyl-1H-pyrrol-2-ylmethylene)-hydrazide; Isonicotinic acid (1-methyl-2-oxo-1,2,dihydro-indol-3-ylidene) hydrazide; Isonicotinic acid (1-methyl-2-oxopropylidene)-hydrazide; Isonicotinic acid (1-P-tolyl-ethylidene)-hydrazide; Isonicotinic acid (1-phenyl-ethylidene)-hydrazide; Isonicotinic acid (1H-indol-2-ylmethylene)-hydrazide; Isonicotinic acid (2,3,4,6-tetramethyl-benzylidene)-hydrazide; Isonicotinic acid (2,4-dihydroxy-benzylidene)-hydrazide; Isonicotinic acid (2,5-dimethoxy-benzylidene)-hydrazide; Isonicotinic acid (2-chloro-benzylidene)-hydrazide; and 1-oxy-isonicotic acid methyl ester.

Quercetin

Quercetin is a member of a group of naturally occurring compounds, the flavonoids, which have common flavone nucleus composed of two benzene rings linked through a heterocyclicpyrone ring.

Quercetin is found in various plants, food products, and dyes of natural origin. The estimated average daily intake of quercetin by an individual in the United States is 25 mg.

The Food and Drug Administration nominated quercetin for toxicity and carcinogenicity studies in the rat because it is a chemical that is widely distributed in foods. Quercetin was administered to rats by dosed feed since human exposure is by dietary consumption.

The Quercetin derivatives and/or analogs that may also be used in the combination of the present invention may be selected from the group consisting of flavone,3,3′,4′,5,7-pentahydroxy-4H-1-benzopyran-4-one;.2-(3,4,-dihydroxypehnyl)-3,5,7-trihydroxy-4H-1-benzopyran-4-one; 3,3′,4′,5,7,-pentahydroxyflavone; 3,5,7,3′,4′-pentahydroxyflavone; 2-(3,4-dihydroxyphenyl)-3,5,7-trihydroxy-4H-1-benzopyran-4-one; 3,5,7,3′,4′-pentahydroxyflavone; 3′,4′,5,7-tetrahydroxyflavan-3-OL; 2-(3,4-dihydroxyphenyl)-4H-1-benzopyran-4-one; cyanidelonon 1522; C.I. natural yellow 10; C.I. natural yellow 10 & 13; C.I. natural red 1; C.I. 75670; meletin; quercetine; quercetol; quercitin; quertine; sophoretin; T-Gelb BZW. Grun 1Xanthaurine; and NCI-C60106.

Quercetin dihydrate analogs may be selected from the group consisting of: Quercetin dihydrate; (±)-Taxifolin; Fisetin; Quercetin-3-rhamnoside; Quercetin-3-D-xyloside; Quercetin-3-D-galactoside; Quercitrin; Rutin trihydrate; Morin; and Morin hydrate.

As mentioned earlier other antioxydant substances may be used in the context of the present invention. For instance, Ginkgo biloba is known for its antioxydant caracteristic for reducing oxidative stress or damage in the brain of AD patients.

Ginkgo Biloba

The Ginkgo biloba extract, obtained from the leaf of a tree of the same name, is a vegetable extract used in Europe to alleviate the symptoms associated with cognositive disorders.

Recently its use has been approved in Germany for the treatment of dementia. Its action mechanism on the central nervous system is only partially known, but its main effects would be related with its antioxidant capacity, diminishing the oxidative stress or damage detected in the brain of patients with Alzheimer's disease. For that purpose, its active principle, characterized as Egb 761, would develop a synergistic action of flavonoids, terpenoids and organic acids that constitute it, that would act synergistically in various processes such as the inflammation homeostasis and the oxidative stress, protecting the membrane and the modulation of the neurotransmission.

Hofferberth et al. studied 36 patients with typical symptoms of psychotic organic syndromes. These patients were divided in two groups, one treated with placebo and the other with EGb 761, 120 mg/day, during a period of 8 weeks.

At the beginning and at the end of the treatment, the patients were subjected to a quantitative electroencephalogram, saccadic eye movements and a psychometric test. Four weeks after the beginning of the treatment, a significant difference could already be observed that persisted until the end of it.

Kanowski et al. performed a multicenter study with 216 patients during 24 weeks divided in a placebo group and a group treated with 240 mg of EGb 761.

The analyzed variables were:

clinic global impression for phyisiopathologic evaluation;

evaluation test of attention and memory; and

evaluation test of daily activities.

Of the total enrolled patients, 156 completed the protocol and, according to Fisher's statistical test, significant improvement was achieved (p<0.005) in the treated group.

LeBars et al. have recently made an important study taking into account the number of patients enrolled and the duration of the treatment. It was a multicenter study with 309 patients of both sexes over 45 years with duration of 52 weeks. In the selection of the cases, dementia was mild to moderate with a score of 9 to 26 carried out according to Mini-mental-Test-Examination (MMSE) and 3 to 6 points according to the Clinical Global Impression of Change, CGIC scale.

The patients received placebo or 120 mg of EGb 761.

The treatment did not show detectable differences in the CGIC, but there was a cognositive significant improvement in the Alzheimers Disease Assessment Scale [ADAS].

A meta-analysis comparing the cholinesterase inhibitors of the tacrine, rivastigmine and metrifonate and the Egb 761, based on published double blind studies of at least 6 months of duration, shared similar effectiveness of both types of compounds evaluated with the ADAS in AD from mild to moderate.

Future investigations are required to specify these mechanisms in connection with the EGb 761, in order to explore the potential of this vegetable extract.

Procedure

2.1. Objective

The effectiveness of the following combination referred to as CP O8T was evaluated:

nicotinic acid: 50 mg;

caffeic acid: 50 mg;

Quercitin: 50 mg;

vitamin E: 1,000 UI504

It was compared to placebo, according to the following design: A double blind comparative trial with cross-over design was used. Patients were divided in 4 groups:

Group 1: patients 1 to 5: they took CPO1P (which corresponds to a placebo cocktail) during 180 days.

Group 2: patients 6 to 9 took CPO1P during 90 days and CPO8T during 90 days.

Group 3: patients 10 to 18: they took CPO8T during 180 days.

Group 4: patients 19 to 22: they took CPO8T during 90 days and CPO1P during 90 days.

2.2. Inclusion Criteria

22 patients with probable Alzheimer's disease were included according to the criteria of the DSM IV (Diagnostic and Statistical Manual Mental of Disorders) and of the NINCDS-ARDRA National Institute of Communicative Disorders and Stroke-Alzheimer's Disease and Related Disorder Association). The selected patients were of both sexes, with ages between 50 and 90 years.

They presented a mild to moderate degree of the disease, which was evaluated with the Mental Mini State of Folstein: they had a score from 10 to 24 points. The Computed Axial Tomography (CAT) or the Nuclear Magnetic Resonance (NMR) did not show infection evidence, infarct or other focal lesions previous to the 12 months of the beginning of the study.

The patients who presented any of the following items were excluded:

1—Knowable hypersensitivity to any of the drugs

2—Not controlled systematic illnesses, such as

cancer in a stage of radio or chemiotherapic treatment

acute myocardal infarctus anger

chronic obstructive lung disease or severe asthma

active gastroduodenal ulcer or recent digestive hemorrhage

severe-anemia (Hb<11), decresase of platelets, acute leukemia

decompensated diabetes

not controlled hypertension

antecedents of severe hepathic illness, acute or chronic hepatopathy

coagulopathy or vitamin K deficit within two years

Neurological diseases such as:

Parkinson disease

Multiinfarct dementia

Huntington disease

Hydrocephalus normotensive

Cerebral-tumor

Progressive supranuclear paralysis

Convulsive-illnesses

Subdural haematoma

Multiple-sclerosis

antecedents of cranial trauma followed by neurological dysfunctions

Psychotic dysfunctions

Patients treated with:

Cholinesterase inhibitors, vasodilators, calcium antagonists, antioxidants or nootropic drugs.

beta blockers, metildope, clonidine

typical neuroleptic sedatives or clozapine

opioids

benzodiazepine: allowed alprazolam up to 1 mg/d

begining of a treatment with antidepressants within 4 weeks

cholinergics or anticholinergics (tricyclic antidepressants included)

corticosteroids

anticonvulsivants

warfarin, and

multivitamins.

All patients and their relatives gave their formal written consent to participate in the study.

The cocktail was administered to the patients. The cocktail was given in person to them, and they were reminded by telephone to take the compounds 3 times a week during the 180 days of the study.

The cognitive subscale of the scale ADAS (Alzheimer's Disease Assesment Scale) was taken previous to the administration of the drug, and 3 and 6 months after the beginning of its administration to evaluate the changes of the cognitive symptoms. The cognitive subscale was then taken again at 9 months, that is to say, 3 months after the interruption of the administration of the compounds.

Evaluation Instruments

1. Mini mental State (Folstein et al, 1975): evaluates orientation, memory, attention, concentration, possibility to name objects, repetition, understanding, ability to make a sentence and to copy two polygons in intersection. The lesser cognitive performance, the lower score. The maximum performance is 30.

2. Alzheimer's Disease Assessment Scale (ADAS) cognitive subsc ale (Rosen, Mohs et al, 1984): evaluates memory, attention, reasoning, language, orientation and praxis. The lesser cognitive performance the higher score. Maximum severity is 70.

3. Hamilton depression scale of 21 items: evaluates mood, feeling of guilt, suicide, insomnia, work and activities, inhibition, agitation, anxiety, somatic symptoms, perspicacity, diurne variation, paranoid symptoms and obsessive-compulsive symptoms.

The higher the score, the greater is the severity. Maximum is 57.

Clinical interviews were carried out previous to the beginning of the study, and then quarterly during the following 9 months.

Complete blood and urine tests were made at the beginning of the study and then quarterly during the following 9 months.

### III. Results

### Statistical Analysis

Patient division according to medication distribution

| | | |
|---|---|---|
| GROUP 1: | CP01P/CP01P | n = 5 |
| GROUP 2: | CP01P/CP08T | n = 4 |
| GROUP 3: | CP08T/CP08T | n = 9 |
| GROUP 4: | CP08T/CP01P | n = 4 |

Evaluation at different periods

| | |
|---|---|
| T 0 M | Before the treatment |
| T 3 M | 3 months |
| T 6 M | 6 months |
| T 9 M | 9 months (3 months after end of treatment) |

Patients that did not present results from any tests were eliminated from the research. Because of this, the groups of treated individuals are not balanced and therefore the means of the groups are used instead of using individual patent date. The statistical study used analysis of variance (ANOVA) using Randomised Block Design (RBD). The RBD allowed to distinguish possible differences between blocks and columns. The block is the random factor. Each block is an individual, or in this case in working with means, a homogeneous group of individuals, who undergo different treatments or different methods or evaluation. Therefore, the research consisted in determining if significant differences existed between the means obtained in the different groups under different treatments and between different time periods of evaluation.

| | T 0 M | T 3 M | T 6 M | T 9 M | |
|---|---|---|---|---|---|
| GROUP 1 | yij | | | | yl. |
| GROUP 2 | | | | | |
| GROUP 3 | | | | | |
| GROUP 4 | | | | | |
| | yj. | | | | y.. |

yij is each data, in the case working with means it is considered the mean of each group in each period of time (box)

yi. is the mean of each block

yj. is the mean of each column

y.. is the general mean

J is the number of trials or evaluations

l is the number of blocks or groups or treatments

The following is the ANOVA table for RBD

| S of V | | | | |
|---|---|---|---|---|
| F | DF | SS | MS | f |
| Between the blocks | I − 1 | J.SUMyl.² − Ny..² | SC_{Ebl}/GL_{Ebl} | CM_{Ebl}/CM_{D} |
| F_{I - 1,GLD,0,05} | | | | |
| Between columns | J − 1 | I.SUMyj.² − Ny..² | SC_{Ecol}/GL_{Ecol} | CM_{Ecol}/CM_{D} |
| FJ − _{1,GLD,0,05} | | | | |
| Error or inside | (I − 1)(J − 1) | SC₁ − (SC_{Ebl} + SC_{Ecol}) | SC_{D}/GL_{D} | |
| Total | N − 1 | SUMyij² − Ny..² | | |

S of V: Source of variation
DF: Degree of freedom,
SS: sum of the squares,
MS: mean of the squares,
SUM: summation.

The result is significant with 95% certainty when f>statistical result of F or, what is the same when p<0,05.

The RBD is an additive model as long as there is no interaction between the blocks and the columns. To make sure of this, each case was tested for non additivity of Tukey.

On the other hand, when significant differences are obtained between blocks or between columns in the ANOVA, contract studies must be carried out to determine between which, of the blocks or the columns, these differences are produced. The contrasts oppose the blocks together or the columns together, and establish the origin of the differences. Contrasts of Scheffe, Newman Keuls and LSD were used.

### Statistical Analysis for the ADAS Test

Table of Means

| | T 0 M | T 3 M | T 6 M | T 9 M | Mean yl. |
|---|---|---|---|---|---|
| GROUP 1 | 41.000 | 44.000 | 42.500 | 45.750 | 43.313 |
| GROUP 2 | 40.800 | 36.800 | 37.800 | 41.800 | 39.300 |
| GROUP 3 | 40.444 | 36.667 | 39.889 | 40.444 | 39.361 |
| GROUP 4 | 40.250 | 36.750 | 38.750 | 39.750 | 38.125 |
| Mean y.j | 40.624 | 38.304 | 39.235 | 41.936 | 40.025 |

### RBD (Randomized Block Design)—ANOVA (Analysis of Varia nce)

| S of V | DF | SS | MS | f | F | |
|---|---|---|---|---|---|---|
| Groups | 3 | 61.5361236 | 20.5120412 | 7.81933039 | >3.88 | S |
| (Blocks) | | | | | | (p < |
| | | | | | | 0.05) |
| Periods | 3 | 30.3659336 | 10.1286445 | 3.88110857 | −3.66 | (p − |
| | | | | | | 0.05) |
| | 9 | 23.6092301 | 2.62324779 | | | |
| Total | 15 | 115.531287 | | | | |

S: Significant differences

NS: Non-significant differences.

The ANOVA reveals significant differences (p<0,05) between the groups or blocks. That is to say between the different treatments as for the average scores obtained for the ADAS test. However, the differences were not as significant for the different times of evaluation.

Contrasts

The different groups were compared to evaluate how their averages differentiate in the ADAS test

Scheffe Test

| | GROUP 1 | GROUP 2 | GROUP 3 | GROUP 4 |
|---|---|---|---|---|
| GROUP 1 | | 0.12010232 | 0.12742966 | 0.0348689 |
| GROUP 2 | 0.120102316 | | 0.99998283 | 0.89121008 |
| GROUP 3 | 0.127429664 | 0.99998283 | | 0.8767038 |
| GROUP 4 | 0.034868896 | 0.89121008 | 0.8767038 | |

GROUP 1 vs 2: NS
GROUP 1 vs 3: NS
GROUP 1 vs 4: S p < 0.05
GROUP 2 vs 3: NS
GROUP 2 vs 4: NS
GROUP 3 vs 4: NS

Newman-Keuls Test

| | GROUP 1 | GROUP 2 | GROUP 3 | GROUP 4 |
|---|---|---|---|---|
| GROUP 1 | | 0.12010232 | 0.12742966 | 0.0348689 |
| GROUP 2 | 0.049438715 | | 0.96941215 | 0.44869167 |
| GROUP 3 | 0.021887839 | 0.96941215 | | 0.69663686 |
| GROUP 4 | 0.021364689 | 0.44869167 | 0.69663886 | |

GROUP 1 vs 2: S p < 0.05
GROUP 1 vs 3: S p < 0.05
GROUP 1 vs 4: S p < 0.05
GROUP 2 vs 3: NS
GROUP 2 vs 4: NS
GROUP 3 vs 4: NS

It can be inferred from observing the graph of the means and the contrasts that the existing significant differences between the treatments are mainly found between groups 1 and 4. Group 1 obtained average scores of ADAS greater than the rest of the groups during the entire time period and moreover, demonstrated a tendency to increase, which indicates a worsening during the treatment. Instead, the other groups average scores decreased during the first 3 months of the treatment but started to increase again from that point on until reaching, in general, the values obtained at the beginning of the study. The Scheffe Test reveals less (is less sensitive) and, for that reason, only demonstrates this difference between groups 1 and 4. On the contrary, the Newman Test demonstrates significant differences between the scores of the groups 1 and 2, 1 and 3, and 1 and 4, resembling the values obtained by the patients of groups 2, 3 and 4 with similar behaviors of their averages during the entire time.

### Statistical Analysis for the MMSE Test

Table of Means +B287

| | T 0 M | T 3 M | T 6 M | T 9 M | Mean yl. |
|---|---|---|---|---|---|
| GROUP 1 | 18.000 | 17.750 | 18.750 | 17.750 | 18.063 |
| GROUP 2 | 19.000 | 19.200 | 20.000 | 18.000 | 19.050 |
| GROUP 3 | 19.000 | 20.333 | 19.222 | 18.778 | 19.333 |
| GROUP 4 | 19.250 | 20.750 | 20.750 | 18.250 | 19.750 |
| Mean y.j | 18.813 | 19.508 | 19.681 | 18.194 | 19.049 y.. |

Behavior of the average scores during the entire time period for the different groups (treatments)+B333

| S of V | DF | SS | MS | F | F | |
|---|---|---|---|---|---|---|
| Groups | 3 | 6.16135882 | 2.08045287 | 6.08060482 | >3.66 | S |
| (Blocks) | | | | | | (p < |
| | | | | | | 0.05) |
| Periods | 3 | 5.58373387 | 1.86124482 | 5.49272112 | <3.88 | S |
| | | | | | | (p < |
| | | | | | | 0.5) |
| | 9 | 3.0497091 | 0.33885857 | | | |
| Total | 15 | 14.8148016 | | | | |

S: Significant differences
NS: Non-significant differences.

In this case, the ANOVA reveals significant differences (p<0,05) between the groups or treatments in their average scores obtained in the MMSE test and also in their different evaluation periods.

Contrasts

On the one hand, the different groups were compared together and, on the other hand, the different periods of evaluation were compared together, using ANOVA, to evaluate how their averages differentiate in the MMSE test. The following tables illustrate the p values of those comparisons:

Scheffe Test

| | GROUP 1 | GROUP 2 | GROUP 3 | GROUP 4 |
|---|---|---|---|---|
| GROUP 1 | | 0.46793833 | 0.26549053 | 0.09729582 |
| GROUP 2 | 0.46793833 | | 0.97262728 | 0.71923995 |
| GROUP 3 | 0.26549353 | 0.97262728 | | 0.92056388 |
| GROUP 4 | 0.09729852 | 0.71923995 | 0.92056388 | |

GROUP 1 vs 2: NS
GROUP 1 vs 3: NS
GROUP 1 vs 4: NS
GROUP 2 vs 3: NS
GROUP 2 vs 4: NS
GROUP 3 vs 4: NS

Newman-Keuls Test

| | GROUP 1 | GROUP 2 | GROUP 3 | GROUP 4 |
|---|---|---|---|---|
| GROUP + | | 0.12574399 | 0.12766111 | 0.06535465 |
| B2541 | | | | |
| GROUP 2 | 0.12574399 | | 0.64535832 | 0.49389362 |
| GROUP 3 | 0.12766111 | 0.64535832 | | 0.50054574 |
| GROUP 4 | 0.06535465 | 0.49389362 | 0.50054574 | |

GROUP 1 vs 2: NS
GROUP 1 vs 3: NS
GROUP 1 vs 4: NS
GROUP 2 vs 3: NS
GROUP 2 vs 4: NS
GROUP 3 vs 4: NS

LSD Test

| | GROUP 1 | GROUP 2 | GROUP 3 | GROUP 4 |
|---|---|---|---|---|
| GROUP 1 | | 0.46793833 | 0.05565747 | 0.01564395 |
| GROUP 2 | 0.12558138 | | 0.6452055 | 0.26582634 |
| GROUP 3 | 0.05565747 | 0.6452055 | | 0.50037628 |
| GROUP 4 | 0.01564395 | 0.26582634 | 0.50037628 | |

GROUP 1 vs 2: NS
GROUP 1 vs 3: NS
GROUP 1 vs 4: S p < 0.05
GROUP 2 vs 3: NS
GROUP 2 vs 4: NS
GROUP 3 vs 4: NS

Scheffe Test

| | T 0 M | T 3 M | T 6 M | T 9 M |
|---|---|---|---|---|
| T 0 M | | 0.74233615 | 0.59628534 | 0.80315143 |
| T 3 M | 0.74233615 | | 0.99412596 | 0.26565802 |
| T 6 M | 0.59628534 | 0.99412596 | | 0.1812412 |
| T 9 M | 0.80315143 | 0.26565802 | 0.1812412 | |

T 0 M vs T 3 M: NS
T 0 M vs T 6 M: NS
T 0 M vs T 9 M: NS
T 3 M vs T 6 M: NS
T 3 M vs T 9 M: NS
T 6 M vs T 9 M: NS

Newman-Keuls Test

| | T 0 M | T 3 M | T 6 M | T 9 M |
|---|---|---|---|---|
| T 0 M | | 0.28400975 | 0.37189716 | 0.33882374 |
| T 3 M | 0.28400975 | | 0.78607857 | 0.12776077 |
| T 6 M | 0.37189716 | 0.78607857 | | 0.13094723 |
| T 9 M | 0.33882374 | 0.12776077 | 0.13094723 | |

T 0 M vs T 3 M: NS
T 0 M vs T 6 M: NS
T 0 M vs T 9 M: NS
T 3 M vs T 6 M: NS
T 3 M vs T 9 M: NS
T 6 M vs T 9 M: NS

LSD Test

| | T 0 M | T 3 M | T 6 M | T 9 M |
|---|---|---|---|---|
| T 0 M | | 0.28388375 | 0.18696201 | 0.33869615 |
| T 3 M | 0.28388375 | | 0.78594285 | 0.05570481 |
| T 6 M | 0.18696201 | 0.76594285 | | 0.03376284 |
| T 9 M | 0.33869615 | 0.05570481 | 0.03376284 | |

T 0 M vs T 3 M: NS
T 0 M vs T 6 M: NS
T 0 M vs T 9 M: NS
T 3 M vs T 6 M: NS
T 3 M vs T 9 M: NS
T 6 M vs T 9 M: S p < 0.05

In regards to the contrasts between the groups of treatments, the Scheffe and Newman-Keuls tests do not succeed in finding differences between those, except in groups 1 vs 4, which demonstrate the smallest value of p, although it is not significant (p<0,05) in either case. However, with a third test (LSD Test), one can see how groups 1 and 4 present this significant difference amongst each other, which was starting to be seen with prior contrasts. On the other hand, one can also see that a p value very close to 0,05 appears when comparing groups 1 vs 3, which also would have markedly different averages between each other. Observing the graph, one can clearly note these differences but also make out the tendencies and the behavior of the means for each of the treatments. Note how the means of the Groups 2, 3 and 4 increase during the first 3 months of the treatment while in Group 1 one can observe a certain decrease during this same period. Then, during the next 3 months, only group 3 shows a decrease in its values and therefore, in the cognitive performance of the patients, while the other groups maintain or increase their scores.

It must be emphasized however that the mean of the initial score for group 1 was less than that of the other groups of the study, fact that must be taken into consideration and without which the behaviour of this group would not differ as much from those of the groups 2 and 4. Finally, all the curves start decreasing from a critical point, marked by the end of the treatments, reaching MMSE score values similar or even less than those obtained at the beginning of the experiment. In regards so the comparison between the different evaluated time periods, again the Scheffe and Newman-Keuls tests do not show differences between those although they present lesser p values at times T 3M vs T 9M and T 6M vs T 9M. Then the LSD test confirms one of the significant differences with p<0,05 and the other with p=0.0557. This means that the values of the averages of the MMSE are significantly different between 6 and 9 months, and, with a somewhat lower significance, between 3 and 9 months.

The graph clearly demonstrates these differences, mostly those which appear between the last two evaluations.

### Statistical Analysis for the Hamilton Test

### Table of Means

| | T 0 M | T 3 M | T 6 M | T 9 M | Mean yl. |
|---|---|---|---|---|---|
| GROUP 1 | 13.75 | 18.25 | 24.75 | 22.5 | 19.813 |
| GROUP 2 | 17.39996 | 15 | 20 | 22.7999992 | 18.800 |
| GROUP 3 | 17.5555553 | 18.1111107 | 24.4444447 | 20.444447 | 20.139 |
| GROUP 4 | 17.250 | 18.5 | 22.25 | 22.25 | 20.063 |
| Mean y.j | 16.489 | 17.465 | 22.861 | 21.999 | 19.703 y.. |

| S of V | DF | SS | MS | f | F | |
|---|---|---|---|---|---|---|
| Groups | 3 | 4.58656281 | 1.52885094 | 0.43474727 | >3.88 | NS |
| (Blocks) | | | | | | |
| Periods | 3 | 122.32563 | 40.7752098 | 11.5948245 | >3.66 | S |
| | | | | | | (p − |
| | | | | | | 0.05) |
| | 9 | 31.6497869 | 3.51664299 | | | |
| Total | 15 | 158.581969 | | | | |

In this case, the ANOVA reveals significant differences (p<0,05) in the average values of the Hamilton test for depression between the different periods of evaluation but not between the groups or treatments.

Contrasts

The different time periods were compared, given the signification of the ANOVA to evaluate how their averages differentiate in the Hamilton test. The following tables demonstrate the p values of these results.

Scheffe Test

| | T 0 M | T 3 M | T 6 M | T 9 M |
|---|---|---|---|---|
| T 0 M | | 0.8872587 | 0.00216968 | 0.0065899 |
| T 3 M | 0.88725287 | | 0.0076508 | 0.02397896 |
| T 6 M | 0.002116968 | 0.0076508 | | 0.91840172 |
| T 9 M | 0.0065899 | 0.02397896 | 0.91840172 | |

T 0 M vs T 3 M: NS
T 0 M vs T 6 M: S p < 0.05
T 0 M vs T 9 M: S p < 0.05
T 3 M vs T 6 M: S p < 0.05
T 3 M vs T 9 M: S
T 6 M vs T 9 M: NS

Newman-Keuls Test

| | T 0 M | T 3 M | T 6 M | T 9 M |
|---|---|---|---|---|
| T 0 M | | 0.44252211 | 0.00127918 | 0.0021444 |
| T 3 M | 0.00127918 | | 0.00248516 | 0.00324839 |
| T 6 M | 0.00127918 | 0.00248516 | | 0.49628913 |
| T 9 M | 0.0021444 | 0.00324839 | 0.49628913 | |

T 0 M vs T 3 M: NS
T 0 M vs T 6 M: S p < 0.05
T 0 M vs T 9 M: S p < 0.05
T 3 M vs T 6 M: S p < 0.05
T 3 M vs T 9 M: S
T 6 M vs T 9 M: NS

It can be inferred from the contrasts that there are significant differences in the means between the time periods of T 0M vs T 6M, T 0M vs T 9M, T 3M vs T 6M and T 3M vs T 9M. Observing the graph of the means, we see how they vary in the time periods mentioned but not between the different groups for each particular period. One can observe how similar the curves are between the groups (ANOVA NS), the means increasing in all cases during the entire time period until the end of the treatment. From that point on, only one of the groups demonstrates an increase while the values of the other groups start to decrease.

Finally, in regards to the ADAS test, the results of group 1 increased during the entire treatment, which would indicate an increase of the severity of the disease. The rest of the groups demonstrated a marked decrease in the three frist months and then increased, in a small or large measure, but without reaching the severity of group 1 (group 4<group 2<group 3). As for the results of the MMSE, once more, group 1 demonstrates a failure to respond to the treatment because weak values are always obtained, while group 4 obtained the best scores, remaining stable in time during the entire treatment. Group 3 improved its scores during the first three months and then they decreased. As for group 2, the scores increased lightly during the 6 months of the experiment.

IV. Intercurrences

Urinary infection

Meningitis

Sudden deaths

Headache

Discussion

In this double-blind, controlled study of patients with Alzheimer's disease, treatment with Nicotinic acid, Caffeic Acid, Quercitin, and Vitamin E combination has proved to be beneficial in delaying disease progression. Disease progression was primarily studied using the standardized tests ADAS and MMSE where longer median time reflects the worsening of symptoms. The median time to the primary outcome was longer with each treatment than with placebo alone. There was a trend toward a delay in reaching each of the individual end points making up the primary outcome, with a significant delay in Alzheimer's Disease Assessment Scale (ADAS) in the treatment Group # 4. There were also significant delays in the deterioration of the performance of activities of daily living and the need for care. These findings should be of interest since, to date, no treatment for Alzheimer's disease has shown similar benefits with respect to these outcomes. The possibility that our findings reflect aberrations in the placebo Group # 1 is unlikely, since the patients in this group reached the end points at the same rate of significance delay in ADAS in the treatment Groups 2 and 3. The Newman Test, demonstrates significant differences between the ADAS score of the Groups 1 and 2, 1 and 3, 1 and 4 resembling the values obtained by the patients of the Groups 2, 3 and 4 with similar behaviors of their averages during the entire period.

There were no demonstrable differences between the results in the group receiving treatment followed by placebo versus the groups receiving treatment throughout the course of the study (Group 2 vs Group 3, Group 2 vs Group 4, and Group 3 vs Group 4).

On the other hand, the ANOVA Test reveals significant differences (p<0,05) between the groups or treatments in their average scores obtained in the MMSE test and also in their different evaluation periods.

The HAMILTON test, which is a measurement of depression, demonstrates that there are significant differences in the means ADAS score between the time periods of T 0M Vs T 6M, T 0M vs T 9M, T 3M vs T 6M and T 3m VS T 9M. The results of group 1 increased during the entire treatment, which indicate an increase in the severity of the disease (demonstrate a failure to respond to the treatment), while group 4 obtained the best ADAS scores, remaining stable in the time during the entire treatment. Group 3 improved its scores during the first three months and then they decreased. As for Group2, the scores increased lightly during the 6 months of the trial.

The above findings suggest that the use of Nicotinic acid, Caffeic Acid, Quercitin, and Vitamin E combination may improve the ADAS scores and delay clinically important functional deterioration in patients with Alzheimer's disease.

Nicotinic acid, Caffeic Acid, Quercitin, and Vitamin E combination may have enhanced the functioning of nigral neurons or enhanced their survival by inhibiting oxidative stress.

In the above study, there was improvement in cognitive portion of the Alzheimer's Disease Assessment Scale and the Mini-Mental State Examination scores in the treatment groups.

The role of Nicotinic acid, Caffeic Acid, Quercitin, and Vitamin E combination in the treatment of neurodegenerative diseases is currently of great interest. Previous trials of alpha-tocopherol at much higher doses than used here, have demonstrated benefit in patients with AD. The neuronal populations involved in Alzheimer's disease are more sensitive to oxidative stress than those in other neurodegenerative diseases. Also, in elderly populations it has been suggested that antioxidants improve cardiovascular function and the immune response and also reduce the risk of cancer. Although it has been found that there is no differences in the frequency of these other types of disease in our study groups, we have no biologic data to evaluate additional possible benefits.

Nicotinic acid, Caffeic Acid, Quercitin, and Vitamin E combination delay functional deterioration, particularly as reflected by the need for institutionalization, and should be considered for use in patients with moderate dementia. Statistically significant results were seen in our model that included adjustment for the baseline differences among the groups in the score on the ADAS test, Mini-Mental State Examination.

Further studies are needed to evaluate patients with more advanced forms of Alzheimer's Disease, and longer observation period to observe any changes in the cognitive scores, behavioral disturbances and functional impairments.

### BIBLIOGRAPHICAL REFERENCES

1. Barkats M, et al. Hopital de la Pitie Salpet riere, France. J Neurochemistry 75: 1438-46; 2000.

2. Behl C, Davis J, Lesley R, et al.: Hydrogen peroxide mediates amyloid b protein toxicity. Cell; 77:817-827; 1994.

3. Behl C, Davis J, Cole G M et al: Vitamin E protects nerve cells fron amyloid beta protein toxicity; Biochem Bi6phis Res Commun, 186:944-950;1992. 5.Benzi G, Moretti A: Are reactive oxygen species involved in Alzheimer's disease; Neurobiology of aging; 16:661-674; 1995.

4. Benzi G., Moretti A:Are reactive oxygen species involved in Alzheimer's disease?; Neurobiology of aging; 16:661-674; 1995

5. Coff J. Natural product formulations available in Europe for psychotropic indication. Psychopharmacol Bull,31 :745-51; 1995.

6. Ehman W D, Ma rkesbery W R, Alauddin M, et al.: Brain trace elements studies in Alzheimer's disease. Neurotoxicology; 7:197-206;1986.

7. Fito M, Covas M I, Lamuela-Raventos R M et al: Protective effect of olive oil and its phenolic compounds against low density lipoprotein oxidation; Lipids, 35(6):633-8;June 2000.

8. Harris M E, Hensley K, Butterfield D A, et al.: Direct evidence of oxidative injury produced by the Alzheimer's b amyloid peptide in cultured hippocampal neurons. Exp Neurol; 131:193-202; 1995.

9. Hensley K, Hall N, Subramanian R, et al.: Brain regional correspondence between Alzheimer1s disease histopatology and biomarkers of protein oxidation; J Neurochem; 65:2146-2156; 1995.

10. Huguet F, Tarrade T. Aipha-adrenoreceptor change during cerebral aging: the effect of Ginkgo biloba extract. J Pharma Pharmacol; 44:24-27;1992.

11. Kanowski S, et al. Proof of efficacy of the ginkgo biloba special extract EGb 761 in outpatients suffering from mild to moderate primary degenerative dementia of the Alzheimer type or multi-infarct dementia. Pharmacopsychiatry; 29:47-56; 1996.

12. Kappus H,Diplock A T: Tolerance and safety of vitamin E: a toxicological position report; Free Radic Biol Med; 13:55-74;1992.

13. Kilander L, Ohrvall M: Alpha tocopherol and Alzheimer's disease; N Engl J Med; 337:572-573; 1997.

14. Le Bars P L, Katz, M, Berman N, et aL A placebo-controlled, double-blind, randomized trial of an extract of Ginkgo biloba for dementia. JAMA; 278:1327-32; 1997.

15. Llesuy S: El proceso de lipoperoxidaci6n. Antioxidantes y Calidad de Vida; 1: 18-19;1994.

16. Lucesoli F, Fraga G C: Evaluaci6n del estre's oxidativo. Antioxidantes y Calidad de Vida; 1:8-13;1995.

17. Marcus D L, Rodirguez T C, Simberkoff K, et al.: Increased peroxidation and reduced antioxidant enzyme activity in Alzheimer's disease. Exp Neurol 150:40-44; 1998.

18. McIntosh U, Trush M A, Troncoso J C. Increased susceptibility of Alzheimer's disease temporal cortex to oxygen free radical-mediated processes. Free Radic Biol Med; 23: 183-190; 1997.

19. Mecocci P, MacGarvey U, Beau M F: Oxidative damage to mitochondrial DNA is increased in Alzheimer's disease. Ann Neurol 1994;36:747-751.

20.Moizeszowicz J, Monczor M: Psicofarmacologia Psicogeria'tnca; in Psicofarmacologia dinamica IV; Buenos Aires;ed Paid6s;1998.

21. Packer L, Haramaki N, Kawabata T, et al. Gingko biloba extract (Egb 761). In Christen Y, Courtois Y, Droy-Lefaix M T, eds. Effect of Gingko biloba Extract (Egb 761) on aging and age-related disorders. Paris. France: Editions Scientifiques Elsevier Paris; 23-47; 1995.

22. Ramassamy C, Avenil D, Theroux L. Oxidative insults are associated with apolipoprotein E genotype in Alzheimer's disease brain. Neurobiol Dis; 7:23-37;2000.

23. Rezvani A H,Levin E D: Cognitive effects of nicotine; Biol Psychiatry,49(3):258-67;Febrary 2001.

24. Sano M, Ernesto C, Thomas R et al: A controlled trial of selegilin e, alpha tocoferol or both as treatment for Alzheimer's disease; N Engl J Med; 336:1216-1222; 1997.

25.Schrattenholz A, Pereira E F, Roth U et al: Agonist responses of neuronal nicotinic acetylcholine receptors are potentiated by a novel class of allosterically acting ligands; Mol Pharmacol; 49: 1-6; 1996.

26. Schippling S, Kontush A, Buhmann C, et al. Increased lipoprotein oxidation in Alzheimerts disease. Free Radic Biol Med; 28:351-360;2000. 33.Schipper H, Sisse S, Stopa E: Expression of heme oxigenase 1 in the senescent and Alzheimer disease; Ann Neurol,37:758-768;1995.

27.Wettstein A: Cholinesterase inhibitors and ginkgo extracts—are they comparable in the treatment of dementia? Comparison of published placebo-controlled efficacy studies of at least six months' duration; Phytomedicine; 6(6):393-401; January 2000.

28.Wevers A, Witter B, Moser N et al: Classical Alzheimer features and cholinergic dysfunctin:towards a unifying hypothesis?;Acta neurol scand Suppi; 176:42-8;2000.

29.Zamani M R, Allen Y S: Nicotine and its interaction with beta-amyloid protein: a short review; Biol Psychiatry,49(3) :221-32 ;February 2001.

1. A pharmaceutical composition for the treatment of cognitive symptoms caused by Alzheimer's disease in a mammal, wherein said composition comprises vitamin E, quercitin, caffeic acid, nicotinic acid or derivatives and/or analogs thereof, and a pharmaceutically acceptable excipient.

2. The pharmaceutical composition of claim 1, wherein the vitamin E is RRR-α-tocopherol.

3. The pharmaceutical composition of claim 1, wherein the quercitin is quescitin dihydrate (3,3′,4′,5,7)-pentahydroxyflavone.

4. The pharmaceutical composition of claim 1, wherein the caffeic acid is (3,4-dihydroxycinnamic) acid.

5. The pharmaceutical composition of claim 1, comprising from 500 UI to 2500 UI of vitamin E.

6. The pharmaceutical composition of claim 5, comprising 1000 UI of vitamin E.

7. The pharmaceutical composition of claim 1, comprising from 0.1 to 100 mg of quercitin.

8. The pharmaceutical composition of claim 7, comprising 50 mg of quercitin.

9. The pharmaceutical composition of claim 1, comprising from 0.1 to 100 mg of caffeic acid.

10. The pharmaceutical composition of claim 9, comprising 50 mg of caffeic acid.

11. The pharmaceutical composition of claim 1, comprising from 0.1 to 100 mg of nicotinic acid.

12. The pharmaceutical composition of claim 11, comprising 50 mg of nicotinic acid.

13. The pharmaceutical composition of claim 1, wherein the mammal is a human.

14. The pharmaceutical composition of claim 1, in the form of a tablet or a capsule.

15. The pharmaceutical composition of claim 1, wherein:
the vitamin E is RRR-α-tocopherol;
the quercitin is quescitin dihydrate (3,3′,4′,5,7)-pentahydroxyflavone; and
the caffeic acid is (3,4-dihydroxycinnamic) acid.

16. The pharmaceutical composition of claim 15, in the form of a tablet or a capsule.

17. The pharmaceutical composition of claim 1, comprising:
from 500 UI to 2500 UI of vitamin E;
from 0.1 to 100 mg of quercitin;
from 0.1 to 100 mg of caffeic acid; and
from 0.1 to 100 mg of nicotinic acid.

18. The pharmaceutical composition of claim 17, in the form of a tablet or a capsule.

19. A method for treating Alzheimer's disease, comprising the step of administering an effective amount of the pharmaceutical composition of claim 1 to a mammal.

20. The method of claim 15, wherein the mammal is a human.
